(19) United States
(12) Patent Application Publication
(10) Pub. No.: US 2016/0331573 A1
(43) Pub. Date: Nov. 17, 2016

(54) TALOTARSAL ORTHOSIS

(71) Applicant: CIZETA MEDICALI FRANCE, St Armand-montrond (FR)

(72) Inventor: Charles DUBOURG, Meillant (FR)

(21) Appl. No.: 14/772,750

(22) PCT Filed: Feb. 24, 2014
(86) PCT No.: PCT/FR2014/050380

§ 371 Date: Oct. 15, 2015

(30) Foreign Application Priority Data
Mar. 12, 2013 (FR) 1352176

(51) Int. Cl. A61F 5/058; A61F 5/01

(52) U.S. Cl. CPC A61F 5/0585; A61F 5/0111

(57) ABSTRACT

The invention relates to an anatomical talotarsal orthosis for immobilising the ankle joint, comprising a plurality of rigid areas (1, 21, 22, 3) lined with a sheath that comes into contact with at least the ankle, and at least one connecting band (4, 5, 6) for attaching the orthosis to the ankle in a detachable and adjustable manner. According to the invention, the purpose of said rigid areas (1, 21, 22, 3) is to immobilise the calcaneus and the cuboid bone.

### TECHNICAL FIELD OF THE INVENTION

The invention relates to the field of rigid orthoses intended to immobilise the ankle and foot, for medical or sporting applications, and/or for persons injured at the ankle.

### PRIOR ART

Known orthoses intended to immobilise the ankle and foot generally consist of a plurality of rigid shells connected to one another by straps or bands that further make it possible to adjust them around the ankle and under the heel. The rigid shells are removable and combined with an under-heel piece that can be set and adjusted.

It is about in all cases holding the ankle in a given fixed position in order for example to stabilise the lateral ligament external to the ankle when it is damaged. The pathologies concerned are: sprains classified as benign to moderate; acute capsuloligamentous injuries on ankle; synovial spills, chronic instability or chronic laxity.

The document WO 1994/005236 discloses an orthosis suited to this type of pathology which comprises an upper region disposed above the ankle and a lower region placed under part of the foot. The regions comprise rigid elements and are connected together by flexible regions and/or fasteners. However, this splint covers the region where an oedema is developed (in the case of a strain), which poses a problem since this causes pain in the wearer of the orthosis. In addition, such an orthosis is relatively complex to manufacture. Furthermore the immobilisation of the malleoli is performed from the bottom of the leg combined with the proximal part of the foot.

The document US 2002/0029009 describes a shell that is relatively complex since it consists of a part to be placed under the foot combined with two lateral flanks connected by straps. Once again the region affected by an oedema is here covered by one of the lateral flanks, which poses a problem. Furthermore and conventionally, this assembly allows immobilisation of the bottom of the leg combined with immobilisation of the proximal part of the foot.

The application GB 2 443 557 is also known which discloses a removable rigid shell whose shape matches that of the posterior part of the ankle, combined with at least one other rigid part intended to be placed on the front of the ankle. In this way a kind of rigid shackle is produced that encloses the ankle and a part of the foot. This type of embodiment is not satisfactory since it is in contact with almost all the entire surface to be immobilised and thereby may create pain in particular on the oedema or oedemas of the patient. Moreover, such a shell being adjusted to the patient, several sizes must be provided in order best to match the shape of the ankle and foot of the patient.

All the known orthoses are based on the combined immobilisation of the bottom of the leg and a part of the foot, which is not satisfactory in particular because they cover and rub any oedemas developed on the patient. In addition, the known orthoses must be worn with a shoe in order to be completely effective. The shoe in fact supplements the immobilisation caused by the splint.

### DISCLOSURE OF THE INVENTION

The invention aims to overcome the drawbacks of the prior art and in particular to propose a novel orthosis (or splint) that can be worn and be effective without a shoe. Moreover the immobilisation provided according to the invention is effected at the heel rather than at the bottom of the ankle and foot.

To do this, it is proposed according to a first aspect of the invention a talotarsal orthosis intended to immobilise the ankle joint, comprising several rigid regions internally covered with a sheath for contact with at least the ankle, at least one connecting band intended to fix the orthosis to the ankle in a removable and adjustable manner. In accordance with the invention, said rigid regions are intended to immobilise the calcaneus and the cuboid bone; and the orthosis comprises two retromalleolar support regions, which leaves clear the two malleoli.

Stabilisation of the calcaneus and cuboid bone are thus achieved; such a locking is effective on the ankle and foot overall; in addition the two malleoli are simultaneously left clear.

Advantageously, leaving clear the lateral malleolus prevents any contact of the orthosis with any oedemas formed close by.

Moreover, the orthosis according to the invention comprises a first rigid region intended to support the soleus. This region thus surrounds the lower rear of the leg while providing an opening at the Achilles tendon.

More precisely, said opening has V shape opening from the rear of the leg to the heel.

Interestingly, the orthosis according to the invention comprises a third rigid region partially covering the lower surface of the foot, from the heel to the cuboid bone. It is here a region intended to support the mid-foot.

According to another feature of the invention, the orthosis comprises at least one connecting band provided with self-gripping surfaces designed to surround the leg circumferentially. Any technically equivalent means may be provided without departing from the scope of the invention.

It consits is a question in all cases of attaching and fixing the orthosis removably on the region concerned.

In accordance with an additional feature of the invention, the orthosis comprises at least one connecting band provided with self-gripping surfaces and designed to surround the dorsal face of the foot. Any technically equivalent means may be provided without departing from the scope of the invention.

Furthermore, said contact sheath covers the entire internal surface of the orthosis. It is intended to make the contact between the orthosis and the skin of the patient more comfortable.

Depending on circumstances, said sheath may have a thickness comprised between about 2 mm and about 6 mm. A person skilled in the art will choose the appropriate thickness. The sheath may be made of PE foam.

Advantageously, said rigid regions consist of PE or another mouldable rigid plastic material. It is therefore particularly easy to manufacture such a rigid shell.

According to a preferred embodiment of the invention, said rigid regions are in a single piece.

### BRIEF DESCRIPTION OF THE FIGURES

Other features, details and advantages of the invention will emerge from the reading of the following description with reference to the accompanying figures which illustrate:

FIG. 1, a perspective view of an orthosis seen from inside, according to one embodiment of the invention;

FIG. 2, a perspective view of an orthosis seen from outside, according to one embodiment of the invention;

FIG. 3, a longitudinal section of an orthosis according to one embodiment of the invention; and

FIG. 4, a rear view of the orthosis.

For more clarity, identical or similar elements are marked by identical reference signs on all the figures.

### DETAILED DESCRIPTION OF AN EMBODIMENT

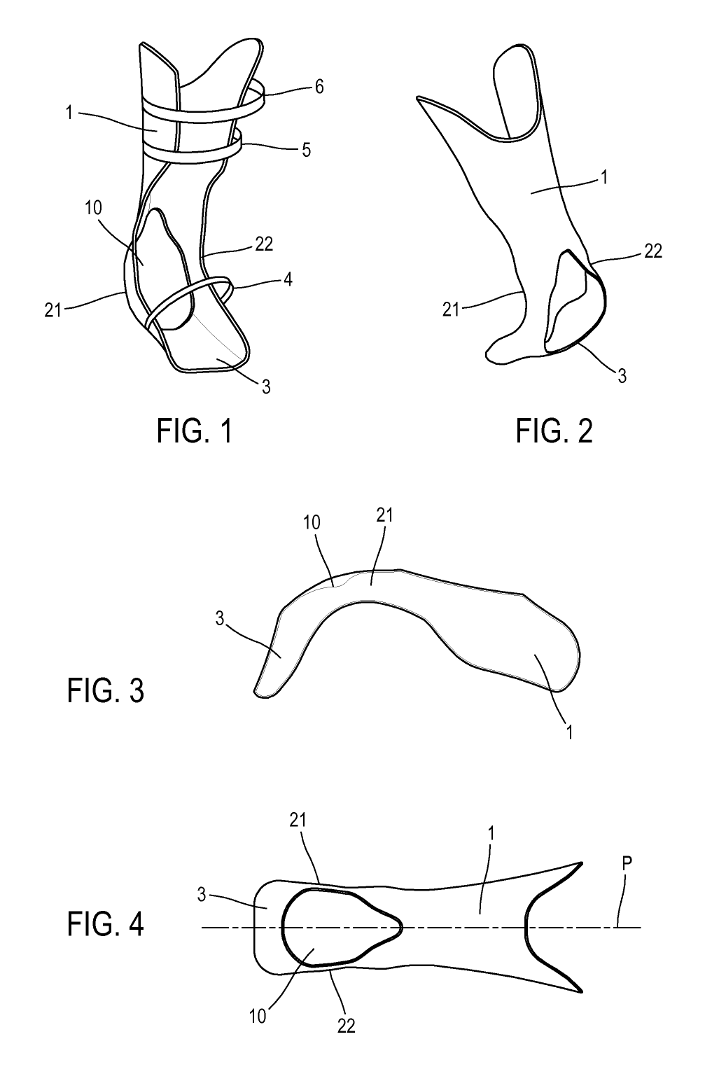

FIG. 1 illustrates an orthosis according to the invention, of anatomical form, intended for example to immobilise an ankle weakened by an impact or inappropriate tension. This orthosis has an anatomical shape that therefore corresponds partially to the external surface of the bottom of the leg and a part of the foot close to the heel. The orthosis comprises several rigid regions 1, 21, 22, 3. It is covered internally by a layer or sheath, for example made of viscoelastic foam (not shown or referenced). This sheath is in direct contact with the skin or a sock or a stocking covering the ankle and/or foot of the patient. The sheath is intended to improve the comfort of the patient since the orthosis is preferentially made of a rigid plastic material such as polyethylene. As will be explained below, such a sheath reduces the pressure points on and around the ankle of the patient. It preferentially covers the entire so-called internal surface of the orthosis. The term “Internal surface” is understood to mean the surface facing and in contact with the ankle to be protected.

Moreover, the orthosis comprises at least one and preferably three connecting bands 4, 5, 6 intended to fix it in a removable and adjustable manner, on and around the ankle.

Preferably, the connecting band is provided with self-gripping surfaces, of the Velcro® type, which makes it possible to adjust it easily on the ankle. Such a band may easily be fitted and removed vis-à-vis the ankle. Any technically equivalent means naturally force within the scope of the invention.

A first connecting band 4 can be provided at the foot, more precisely with the dorsal face of the foot. It cooperates with one of the rigid regions, as will be explained below.

One or more other rigid bands 5, 6 can cooperate with another region of the orthosis in order to surround the calf according to its circumference.

Means for connection between the bands 4, 5, 6 and the rigid regions are provided, known per se.

Particularly advantageously, the anatomical orthosis according to the invention comprises rigid regions that are intended to immobilise the calcaneus and the cuboid bone. This is a novel and inventive way of immobilising an ankle. The orthosis is substantially symmetrical vis-à-vis a plane passing through the tibia and the middle of the heel.

More precisely, a first rigid region 1 is provided; it is intended to support the lower rear of the leg, at the calf. In other words, the first region constitutes a rigid support for the soleus. The first region 1 advantageously has a semicylindrical shape, with preferably two projections towards the knee in order better to surround the concerned region. In addition the first region 1 has an opening 10 at the Achilles tendon. More precisely the opening 10 has a V shape that opens towards the heel. The heel is thus left clear, not covered by the orthosis, which avoids injuring it.

The orthosis according to the invention further comprises two retromalleolar support regions 21, 22. These two regions 21, 22 are therefore disposed symmetrically vis-à-vis the symmetry plane of the orthosis and are each intended to cover a region posterior to (that is to say at the rear of) the malleolus, respectively at the median malleolus and the lateral malleolus. “To the rear of” must be understood as in the opposite direction to “in front of”, which corresponds to the phalanges of the foot. These two regions moreover define the limits of the opening 10 and constitute a connection between the first region 1 and a third region 3 supporting the mid-foot.

As its name indicates, the third region 3 supports the foot, from below, under the calcaneus and the cuboid bone. In other words the third region partially covers the lower surface of the foot, from the heel to the cuboid bone.

A person skilled in the art will choose the appropriate dimensions corresponding for example to two or three average morphologies: child, small adult, large adult. The bands 4, 5, 6 and optionally the thickness of the contact sheath will make it possible to more precisely adapt the orthosis to all morphologies. By way of example, the largest dimension of the orthosis, or height, measured along the symmetry plane P, may be comprised between 20 cm and 30 cm. This corresponds to the distance between the ground and half of the calf.

FIG. 3 is a cross section through the symmetry plane P of the orthosis and shows clearly the various rigid regions and their respective arrangements.

FIG. 4, a rear view of the orthosis, shows its symmetry vis-à-vis the plane P. The opening 10 is clearly visible therein.

The various regions 1, 21, 22, 3 may constitute a single rigid shell, moulded in a single piece. They may be formed by two rigid shells assembled and connected by the rear (on the heel side therefore). The thickness of the shell is around three millimetres, for example more or less two millimetres. It may be made of polyethylene or any mouldable rigid plastic material.

Another advantageous feature of the invention lies in the fact that the orthosis can be worn as such, without a shoe, while remaining completely effective. This proves to be very advantageous since it simplifies re-education.

1. An anatomical talotarsal orthosis intended to immobilise the ankle joint, comprising several rigid regions internally covered with a sheath for contact with at least the ankle, at least one connecting band intended to fix the orthosis removably and adjustably to the ankle, wherein said rigid regions are intended to immobilise the calcaneus and the cuboid bone and said rigid regions comprise two retromalleolar support regions that leave clear the two malleoli.

2. An orthosis according to claim 1, further comprising a first rigid region, for supporting the soleus, intended to surround the lower rear of the leg while providing an opening at the Achilles tendon.

3. An orthosis according to claim 2, wherein said opening has V shape opening from the rear of the leg to the heel.

4. An orthosis according to claim 2, further comprising a third rigid region, for supporting the mid-foot, partially covering the lower surface of the foot, from the heel to the cuboid bone.

5. An orthosis according to claim 1, wherein said at least one connecting band, is provided with self-gripping surfaces and is designed to surround the leg circumferentially.

6. An orthosis according to claim 1, wherein said at least one connecting band is provided with self-gripping surfaces and is designed to surround the dorsal face of the foot.

7. An orthosis according to claim 1, wherein said contact sheath covers the entire internal surface of the orthosis.

8. An orthosis according to claim 7, wherein said sheath has a thickness between about 2 millimetres and about 6 millimetres.

9. An orthosis according to claim 1, wherein said rigid regions consist of PE or another mouldable rigid plastic material.

10. An orthosis according to claim 1, wherein said rigid regions are in a single piece.
